SEQUENCE LISTING (4 sequences)

SEQ ID NO: 1 - DNA, 22 bp, synthetic construct
  source 1..22: mol_type = other DNA
cggaccaatacgaccaaatccg

SEQ ID NO: 2 - DNA, 20 bp, synthetic construct
  source 1..20: mol_type = other DNA
agccacatcgctcagacacc

SEQ ID NO: 3 - DNA, 23 bp, synthetic construct
  source 1..23: mol_type = other DNA
tcttagaacaaattctgcccttt

SEQ ID NO: 4 - DNA, 21 bp, synthetic construct
  source 1..21: mol_type = other DNA
tgctttggtcttgaaatcaca

(19) United States
(12) Patent Application Publication
(10) Pub. No.: US 2026/0028589 A1
(43) Pub. Date: Jan. 29, 2026

(54) USE OF HUMAN PLATELET-APOPTOTIC VESICLES

(71) Applicant: PEKING UNIVERSITY SCHOOL AND HOSPITAL OF STOMATOLOGY, Beijing (CN)

(72) Inventors: Yongsheng ZHOU, Beijing (CN); Xiao ZHANG, Beijing (CN); Yuhe JIANG, Beijing (CN); Yuzi SHAO, Beijing (CN); Kunkun YANG, Beijing (CN)

(21) Appl. No.: 18/855,671

(22) PCT Filed: Sep. 9, 2022
(86) PCT No.: PCT/CN2022/117997

§ 371 Date: Oct. 10, 2024

(30) Foreign Application Priority Data
Mar. 8, 2022 (CN) 20221021797.5

(51) Int. Cl. C12N 5/077; A61K 35/19; A61K 47/34; A61P 19/08; C12N 5/078

(52) U.S. Cl. CPC C12N 5/0654; A61K 35/19; A61K 47/34; A61P 19/08; C12N 5/0644; C12N 2501/48; C12N 2506/1353; C12N 2533/40

(57) ABSTRACT

Provided is the use of human platelet-derived apoptotic vesicles in the preparation of a formulation for promoting osteogenic differentiation of mesenchymal stem cells. Further provided is a bone defect repair formulation, comprising a PLGA scaffold material, wherein the human platelet-apoptotic vesicles are loaded on the surface of the PLGA scaffold material.

### REFERENCE TO AN ELECTRONIC SEQUENCE LISTING

The contents of the electronic sequence listing (SequenceListing.xml; Size: 4,592 bytes; and Date of Creation: Aug. 18, 2025) is herein incorporated by reference.

### TECHNICAL FIELD

The present application relates to the technical field of biological tissue engineering, and particularly relates to the use of human platelet-derived apoptotic vesicles.

### BACKGROUND

Apoptosis is a process of highly regulated cell death where specific cells are sacrificed to obtain greater benefits of organisms, and is a normal physiological process of multicellular organisms. Apoptosis makes multicellular organisms have more advantages in a coordinated manner, enabling them to maintain homeostasis and fine-tune their life cycles. Cell death caused by apoptosis undergoes several stages: first, nuclear chromatin is condensed; then membrane vesicles are ruptured; and finally, cellular contents are disintegrated into different membrane-wrapped vesicles, called apoptotic bodies or apoptotic vesicles (apoVs). The apoVs are different from exosomes (a.k.a. exosomal vesicles or efflux bodies), microvesicles (a.k.a. microparticles) and retrovirus-like particles (RLPs). The exosomes, microvesicles and RLPs are secreted in the normal cellular process, while the apoVs are formed only in the programmed cell death process. In the process of apoptosis, contents are polymerized to form the apoVs and are eventually engulfed by phagocytes. This process is known as efferocytosis. Traditionally, efferocytosis is deemed as an end of apoptosis, so that the life of apoptotic cells comes to an end biochemically. However, more and more evidences show that substances packaged in the apoVs are transferred between cells, recycled and even reused.

In the prior art, the apoVs are usually extracted from animal cells or commercial cells. However, the apoVs from animal cells are not isogenous, and may cause immunological rejection in vivo, which also goes against ethical requirements. In contrast, commercially passaged mesenchymal stem cells (MSCs) usually inevitably undergo phenotypic, functional and more important genetic changes, which, compared with primary cells, will lead to unpredictable safety problems, and also affect the osteogenic potential of their apoVs. At present, masses of cells can be extracted from human blood in a safe and convenient manner. Use of cell products derived from homologous blood makes it possible to avoid immunological rejection and unstable effects caused by passage, and is more conducive to clinical applications in the future.

Bone defect is one of the common orthopedic diseases, and common causes include high-energy trauma and disorders, tumor resection, osteomyelitis, developmental malformation, and the like. The human body can repair minor bone defects through remodeling, but prognosis for severe bone defects is usually poor. Large-area bone defects might cause poor bone healing, healing deformities and even pathological fractures. The current gold standard for treatment of bone defects is the use of autogenous bone grafts. However, sources of grafts are limited and donor sites are prone to infection, thereby limiting the large-scale use of autologous bone grafts. With an increasing clinical demand, bone grafting has become the second most common type of tissue transplantation, which is second only to blood transfusion in frequency. In order to solve the problem of shortage of autologous bone, a variety of repair materials, such as allogeneic bone, heteroplastic bone, demineralized bone matrix (DBM), bioceramics, metal scaffolds, polymer scaffolds and other materials, have been used to repair bone defects. Synthetic polymer materials are highly favored due to their mechanical processability, high controllability of degradation, and structural uniformity. Among such materials, polylactic acid-hydroxyacetic acid copolymer (poly(lactic-co-glycolic) acid, PLGA) is most widely used. However, the PLGA scaffolds have the shortcomings of surface hydrophobicity and low osteogenic and angiogenic activities, which limit regenerative stimulation of large-area bone defects.

In order to alleviate the above shortcomings, it is of great significance to develop a safe and effective carrier of biologically active substances and enhance the osteogenic effect of PLGA for bone defect repair.

### SUMMARY

In view of the problems and unsatisfactory effects of existing bone defect repair therapies, a kind of safe and efficient apoptotic vesicles (apoVs) that promote bone regeneration is provided in some embodiments of the present application, with an aim to improve effects of the existing bone defect repair therapies and advance their therapeutic applications.

The present application provides a use of human platelet-derived apoptotic vesicles in the preparation of a formulation for promoting osteogenic differentiation of mesenchymal stem cells.

In some embodiments, a method for preparing the human platelet-derived apoptotic vesicles includes the following steps:
isolating and purifying the human platelets in vitro;
re-suspending the human platelets in a culture solution, adding staurosporine to induce apoptosis of the human platelets; and
collecting a supernatant, and isolating by gradient centrifugation to obtain apoptotic vesicles.

In some embodiments,
a. the culture solution used for re-suspending is a minimum essential medium α (MEMα) containing 10% fetal bovine serum (FBS) and 1% penicillin-streptomycin dual antibiotics;
b. the gradient centrifugation method comprises the following steps:
   centrifuging the supernatant at 4° C. and 800 g for 10 min, to obtain a first centrifugation supernatant;
   centrifuging the first centrifugation supernatant at 4° C. and 16,000 g for 30 min, and taking a precipitate to obtain a second centrifugation supernatant to obtain crude apoVs; and
   washing the crude apoVs with sterile phosphate-buffered saline (PBS), and then centrifuging at 4° C. and 16,000 g for 30 min to obtain pure human platelet-derived apoptotic vesicles; or
c. the apoVs are biconcave and disc-shaped, with a particle size of 90-300 nm.

In some embodiments, the formulation for promoting osteogenic differentiation of mesenchymal stem cells may be a formulation used for bone defect repair.

The present application further provides a bone defect repair formulation, including a polylactic-co-glycolic acid (PLGA) scaffold material, where the human platelet-derived apoptotic vesicles are loaded on a surface of the PLGA scaffold material.

In some embodiments, a process of preparing the bone defect repair formulation includes the following steps:
soaking the PLGA scaffold in a dopamine solution to form a polydopamine (pDA) membrane;
removing unattached excess dopamine molecules to obtain a PLGA/pDA scaffold; and
soaking the PLGA/pDA scaffold in a solution of human platelet-derived apoptotic vesicles.

In some embodiments,
1) the PLGA scaffold has a diameter of 4 mm and a height of 2 mm;
2) soaking the PLGA scaffold in the dopamine solution, and shaking and culturing at 37° C. for 18 h to form a pDA membrane;
3) cleaning with distilled water in an ultrasonic cleaning machine to remove unattached dopamine molecules until the water becomes clear; and after sterilizing with 75% ethanol for 1 h, washing with the sterile PBS for 3 times;
4) a concentration of the dopamine solution is 2 mg/mL, 10 mM of a Tris(hydroxymethyl)aminomethane hydrochloride (Tris-HCl) buffer is used for dissolving, and a pH value is 8.5; or
5) the concentration of the solution of human platelet-derived apoptotic vesicles is 0.5 μg/μL.

The present application further provides a method for preparing the bone defect repair formulation, and the method includes the step of loading the human platelet-derived apoptotic vesicles on a surface of the PLGA scaffold material.

In some embodiments, the preparation method includes the following steps:
soaking the PLGA scaffold in a dopamine solution to form a polydopamine (pDA) membrane;
removing unattached excess dopamine molecules to obtain a PLGA/pDA scaffold; and
soaking the PLGA/pDA scaffold in a solution of human platelet-derived apoptotic vesicles.

In some embodiments,
1) the PLGA scaffold has a diameter of 4 mm and a height of 2 mm;
2) soaking the PLGA scaffold in the dopamine solution, and shaking and culturing at 37° C. for 18 h to form a pDA membrane;
3) cleaning with distilled water in an ultrasonic cleaning machine to remove unattached dopamine molecules until the water becomes clear; and after sterilizing with 75% ethanol for 1 h, washing with the sterile PBS for 3 times;
4) a concentration of the dopamine solution is 2 mg/mL, 10 mM of a Tris(hydroxymethyl)aminomethane hydrochloride (Tris-HCl) buffer is used for dissolving, and a pH value is 8.5; or
5) the concentration of the solution of human platelet-derived apoptotic vesicles is 500 ng/μL.

Some technical solutions of the present application have the following beneficial effects:

In some embodiments of the present application, the human platelet-derived apoptotic vesicles are provided, and the human platelets can be extracted from a patient's own blood, which avoids ethical and immune problems. In some embodiments of the present application, the method for preparing the apoVs is simple and low-cost, enabling a high yield and rapid detection. Use of the apoVs in vitro can promote the osteogenic differentiation of mesenchymal stem cells.

In some embodiments of the present application, a bone defect repair formulation prepared by loading the human platelet-derived apoptotic vesicles on the PLGA/pDA scaffold is disclosed, which overcomes the shortcomings of the PLGA such as hydrophobicity and low osteogenic activity. In particular, by first loading a layer of pDA membrane on the surface of the PLGA scaffold and then loading the human platelet-derived apoptotic vesicles, the present application enables the human platelet-derived apoptotic vesicles to be slowly released in vivo, which further improves the osteogenic capability of mesenchymal stem cells in vivo. Animal in-vivo experiments prove that the bone defect repair formulations in some embodiments of the present application can significantly improve the osteogenic capability of mesenchymal stem cells in vivo and promote bone tissue regeneration without obvious side effects, and have broad clinical application prospects in the field of bone defect repair.

### BRIEF DESCRIPTION OF THE DRAWINGS

In order to more clearly describe the technical solutions in the embodiments of the present application or in the prior art, a brief introduction to the accompanying drawings required for the description of the embodiments or the prior art will be made below. Apparently, the accompanying drawings in the following description are merely some embodiments of the present application, and those of ordinary skill in the art would also be able to derive other drawings from these drawings without making creative efforts.

FIG. 1 is a schematic diagram of a process of inducing apoptosis in vitro through human platelets (PLTs), extracting apoptotic vesicles (apoVs), and constructing a polylactic acid-hydroxyacetic acid copolymer (PLGA)/polydopamine (pDA) scaffold loaded with the PLT-apo Vs.

FIG. 2 is a transmission electron microscope diagram of results of extracting the apoVs, where A is one disc-shaped side of the apoVs, and B is the other disc-shaped side of the apo Vs.

FIG. 3 illustrates detection results of nanoparticle tracking analysis.

FIG. 4 illustrates human platelet-derived apoptotic vesicles (PLT-apoVs) that promote the osteogenic differentiation of human bone marrow mesenchymal stem cells in vitro, where A is a diagram of staining with a proliferation medium (PM) alizarin red, B is a diagram of staining with an osteogenic medium (OM) alizarin red, and C is a diagram of staining with OM+100 ng/mL PLT-apoVs alizarin red.

FIG. 5 is a quantitative diagram of alizarin red.

FIG. 6 illustrates reverse transcription-polymerase chain reaction (RT-PCR) results of human platelet-derived apoVs that promote a runt-related transcription factor 2 (RUNX2) expression of a key gene of osteogenesis.

FIG. 7 is a micro-computed tomography (CT) scan diagram of human platelet-derived apoVs that promote bone defect repair of mesenchymal stem cells.

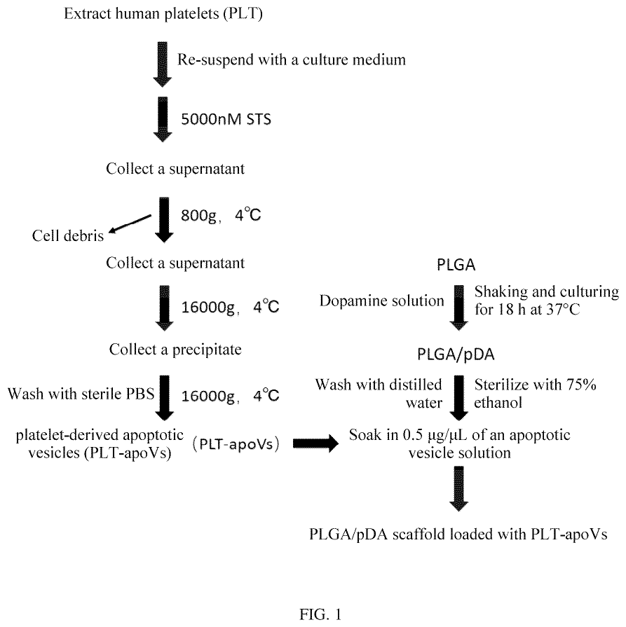

### DETAILED DESCRIPTIONS OF THE EMBODIMENTS

Unless otherwise specified, experimental methods used in the following embodiments are conventional methods.

Unless otherwise specified, the materials, reagents and so forth used in the following embodiments can be obtained commercially.

Unless otherwise specified, percentages used in the following embodiments are mass percentages.

In order to make the technical problems, technical solution and advantages of the present application clearer, detailed description will be made below with reference to the accompanying drawings and specific embodiments.

### Example 1 Efficient Extraction of Human Platelet-Derived Apoptotic Vesicles

The human platelets were isolated and purified in vitro and re-suspended in a culture medium, and 5,000 nM of staurosporine (STS) was added for apoptosis induction. The human platelet-derived apoVs were extracted by gradient centrifugation, and their concentration was detected through nanoparticle tracking analysis. The Bicinchoninic Acid (BCA) protein assay was used to detect a protein content, so as to obtain optimal extraction conditions and establish a standard extraction process.

The process is detailed as follows:
a) a cell culture supernatant was centrifuged at 4° C. at 800 g for 10 min to remove cell debris in the cell culture supernatant, so as to obtain a first centrifugation supernatant;
b) the first centrifugation supernatant was centrifuged at 4° C. and 16,000 g for 30 min, and a precipitate was taken to obtain crude apoptotic vesicles; and
c) the crude apoptotic vesicles were washed with sterile phosphate-buffered saline (PBS), and then centrifuging at 4° C. and 16,000 g for 30 min to obtain pure apoptotic vesicles.

### Example 2 Characteristic Analysis of Human Platelet-Derived Apoptotic Vesicles

The morphology, particle size and concentration of the platelet-derived apoVs were detected through cryogenic transmission electron microscopy and nanoparticle tracking analysis.

### Cryogenic Transmission Electron Microscopy:

(1) 5 μl of an apoptotic vesicles suspension was taken, dropped onto a copper grid, and kept standing at room temperature for 1 min;
(2) excess liquid was absorbed by using filter paper along an outer side of the copper grid, and 5 μl of 2% uranyl acetate was taken and dropped onto the copper grid, and kept standing at room temperature for 30 sec;
(3) excess liquid was absorbed by using filter paper along the outer side of the copper grid, and standing at room temperature was performed to dry; and
(4) images were taken through a transmission electron microscope with a voltage set as 120 kV.

### Detection Through Nanoparticle Tracking Analysis:

(1) a nanoparticle tracking analyzer was used to record the trajectory of the apoVs in the Brownian motion process; and
(2) Nanoparticle tracking analysis (NTA) software was used for analysis.

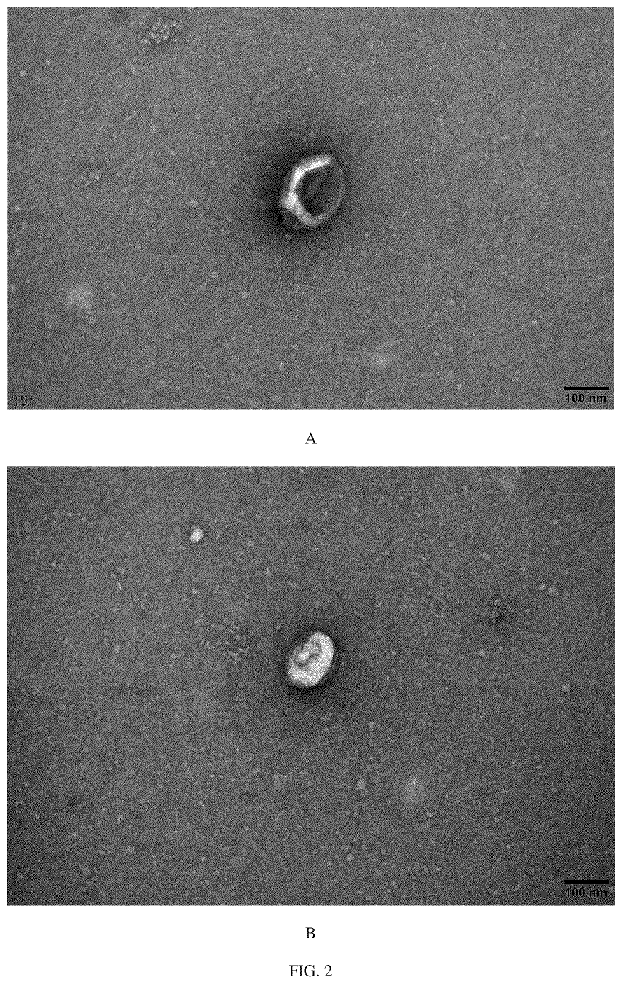

Transmission electron microscope results shown in A and B of FIG. 2 show that the human platelet-derived apoVs are biconcave and disc-shaped.

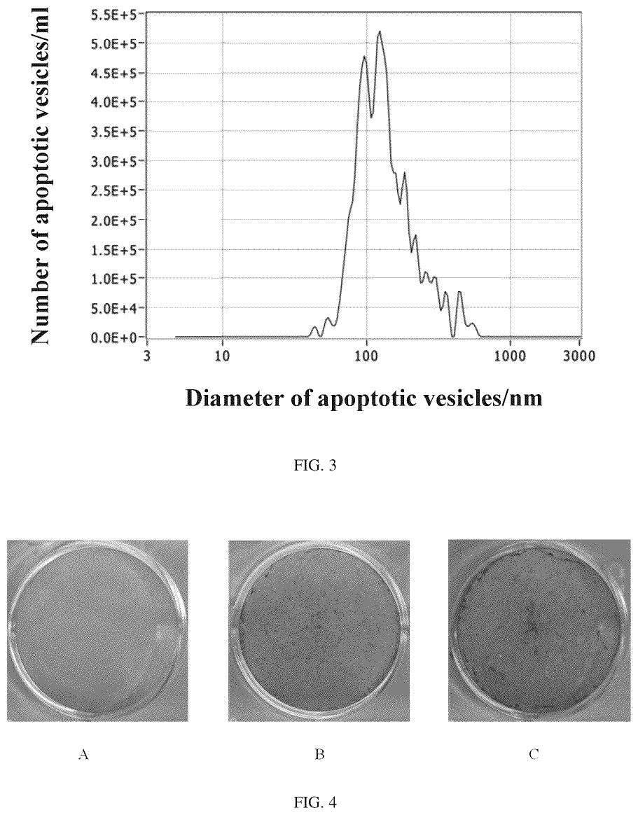

Nanoparticle tracking analysis and detection results in FIG. 3 show that the human platelet-derived apoVs have particle sizes of mainly about 90-300 nm, and an average particle size of 122.7 nm.

### Example 3 Experimental Effect of Human Platelet-Derived Apoptotic Vesicles on the Osteogenic Differentiation of Human Bone Marrow Mesenchymal Stem Cells In Vitro

Human bone marrow mesenchymal stem cells were cultured under the following three culturing conditions:
1) A proliferation medium (PM): a minimum essential medium α (MEMα) containing 10% fetal bovine serum (FBS) and 1% penicillin-streptomycin dual antibiotics.
2) An osteogenic medium (OM): an MEMα containing 10% FBS, 1% penicillin-streptomycin dual antibiotics, 10 mM of β-Sodium Glycerophosphate, 0.2 mM of L-ascorbic acid, and 100 nM of dexamethasone.
3) 100 ng/ml of human platelet-derived apoVs were added to the OM (OM+100 ng/mL PLT-apoVs).

After 10 days of osteogenic induction, the effect of osteogenic differentiation of cells was detected by staining with alizarin red.

### Alizarin Red Staining:

To prepare a dye solution, 1 g of powder (Alizarin red S, SIGMA, A5533-25G) was weighed and dissolved in 100 mL of Milli-Q water.

The culture medium was aspirated, rinsing with phosphate-buffered saline (PBS) was performed for three times, cells were fixed with 95% ice ethanol for 30 min, the ice ethanol was discarded, rinsing with the Milli-Q water was performed for three times, the dye solution was added after drying to dye mineralized nodules, the dye solution was absorbed after the staining, distilled water was used for rinsing, and images were taken under the microscope.

### Quantification by Alizarin Red:

An equal amount of a 1% cetylpyridinium chloride solution was added, 100 μl of the solution was taken into a 96-well plate after complete dissolving, absorbance was measured at a wavelength of 490 nm, and a quantitative analysis of staining with alizarin red was made.

The results of staining with alizarin red shown in FIG. 4: after 10 days of culturing, a large number of red-stained mineralized nodules were generated in the OM (compared with the ordinary PM). When the platelet-derived apoVs were added during culturing in the OM, mineralized nodules of cells significantly increased than those generated in the control group (OM). This indicates that after the apoVs were added, the osteogenic differentiation capability of human bone marrow mesenchymal stem cells in vitro was enhanced.

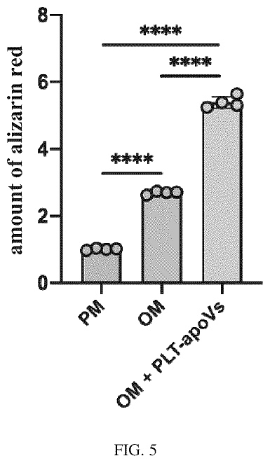

As shown in FIG. 5, corresponding quantitative analysis results of alizarin red are consistent with the staining results (**** p<0.0001).

### Example 4 In Vitro Experiments to Detect an Effect of Human Platelet-Derived Apoptotic Vesicles (PLT-apoVs) in Promoting a Runt-Related Transcription Factor 2 (RUNX2) Expression of a Key Gene of Osteogenesis in Human Bone Marrow Mesenchymal Stem Cells

Cells were inoculated in 6-well plates, and the PLT-apoVs (OM+100 ng/ml PLT-apoVs) were cultured in an ordinary proliferation medium (PM), an osteogenic medium (OM), and other osteogenic induction conditions, respectively. Ribonucleic acid (RNA) was extracted after 10 days of culturing, and reverse transcription-quantitative polymerase chain reaction (RT-PCR) was used to detect a RUNX2 expression of an osteogenesis-related gene.

### (1) Extraction of Total Cellular RNA

Cells were inoculated in the 6-well plates by experimental groups, and the RNA was extracted after induction under different conditions. Specific steps are as follows:
1) cleaning the culture medium with phosphate-buffered saline (PBS);
2) a Trizol reagent (1 mL/well) was added and transferred to a 1.5 mL centrifuge tube;
3) 200 μl of trichloromethane was added, and a mixture was shaken for 30 sec and then placed on ice for 3 min;
4) the mixture was centrifuged at 4° C. and 12,000 g for 15 min;
5) after standing for a while, an upper aqueous phase was transferred to another centrifuge tube;
6) an equal volume of isopropanol was added, and the centrifuge tube was inverted for mixing well, and kept standing on ice for 10 min;
7) the centrifuge tube was centrifuged at 4° C. and 12,000 g for 10 min;
8) a supernatant was discarded, 1 mL of 75% ethanol prepared with pre-cooled anhydrous ethanol and diethyl pyrocarbonate (DEPC) water, and a precipitate was washed;
9) the centrifuge tube was further centrifuged 4° C. and 7,500 g for 5 min; and
10) the supernatant was discarded, liquid was aspirated, the precipitate was dried at room temperature, an appropriate amount of DEPC water was added, and the extracted total RNA was stored in aliquots at −80° C. for storage or for the next experiment.
   (2) Reverse Transcription to Synthesize Complementary Deoxyribonucleic Acid (cDNA)
1) 20 μl of a reverse transcription reaction system was used, and about 1000 ng of total RNA was used;
2) 1,000 ng of extracted total RNA was taken, and a reverse transcription reaction solution was prepared on ice according to kit instructions; and
3) reverse transcription reaction conditions: 37° C., 15 min (a reverse transcription reaction); 85° C., 5 sec (inactivation reaction of reverse transcriptase); maintained at 4° C. (can be stored in aliquots at −20° C. for storage or for the next experiment).

### (3) Real-Time Quantitative PCR Reaction

1) three secondary wells were tested for each sample and each gene, and 20 μl of the reaction system was configured for each well of eight-tube strips. The reagents used and their dosage were as follows, and a primer sequence was shown in Table 1:

| | | |
|---|---|---|
| SYBR Green | 10 | μl |
| cDNA | 0.5 | μl |
| Primers | 1 | μl |
| DEPC water | 8.5 | μl |

2) PCR reaction conditions: hot starting at 95° C. for 10 min, denaturation at 95° C. for 30 sec; annealing and extension at 60° C. for 1 min, a total of 40 cycles; and
3) with glyceraldehyde-3-phosphate dehydrogenase (GAPDH) as an internal reference, a ΔΔCt method was used to analyze the data, and experimental data were expressed by a mean of results of three independent experiments (± a standard deviation).

TABLE 1
RT-qPCR primer sequences

| Gene | SEQ ID NO: | Primer sequences |
|---|---|---|
| GAPDH | 1 | CGGACCAATACGACCAAATCCG |
| | 2 | AGCCACATCGCTCAGACACC |
| RUNX2 | 3 | TCTTAGAACAAATTCTGCCCTTT |
| | 4 | TGCTTTGGTCTTGAAATCACA |

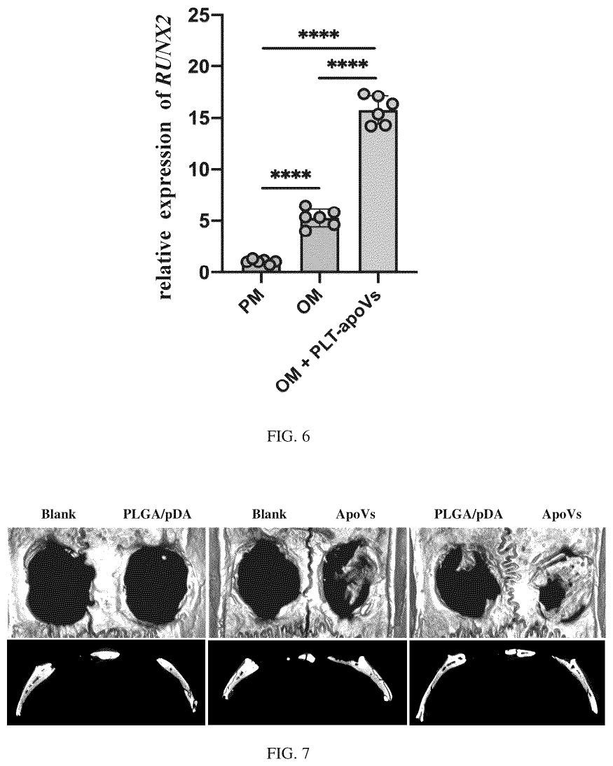

FIG. 6 illustrates the RT-qPCR detection results of upregulating a RUNX2 expression of a key gene of platelet-derived apoVs that promote osteogenic differentiation of human bone marrow mesenchymal stem cells. It can be seen from the figure that after the osteogenic induction, a level of RUNX2 expression was upregulated, and after the apoVs were added, the level of RUNX2 expression was significantly higher than that of the control group (**** p<0.0001), proving that the osteogenic differentiation capability of human bone marrow mesenchymal stem cells in vitro was enhanced.

### Example 5 Experimental Results about In Situ Repair of Rat Skull Defects Prove that Human Platelet-Derived Apoptotic Vesicles Promote Osteogenic Differentiation of Mesenchymal Stem Cells and Bone Defect Repair

(1) Constructing Polylactic Acid-Hydroxyacetic Acid Copolymer (PLGA)/Polydopamine (pDA) Scaffold Loaded with the PLT-apoVs
1) A cylindrical PLGA (lactide/glycolide: 50/50) scaffold was prepared, with a diameter of 4 mm, and a height of 2 mm;
2) the PLGA scaffold was soaked in 2 mg/mL of a dopamine (DA) solution (dissolved in 10 mM of a Tris(hydroxymethyl)aminomethane hydrochloride (Tris-HCl) buffer, with a pH value of 8.5), and shaking and culturing were performed at 37° C. for 18 h to form a pDA membrane;
3) the scaffold was cleaned with distilled water in an ultrasonic cleaning machine to remove unattached DA molecules until the water became clear;
4) after sterilizing with 75% ethanol for 1 h, the scaffold was washed with the sterile phosphate-buffered saline (PBS) for 3 times to obtain a PLGA/pDA scaffold; and
5) the PLGA/pDA scaffold was soaked in 0.5 μg/μL of a PLT-apoVs solution.

### (2) Animal Experiments

1) 9 male SD rats with an average weight of 250-300 g were selected, and randomly divided into three groups, with three rats in each group; animals were housed in a standard room where temperature and humidity were controllable, with a 12-hour light-dark cycle; and all animal experiments were approved by Institutional Animal Care and Use Committee of Peking University Health Science Center and carried out in accordance with institutional animal guidelines;
2) constructing an in situ skull defect model: under the condition that a large amount of physiological saline was used for washing, a trephine (Hager Meisinger GmbH) was used to drill at low speed to construct a skull defect model with a diameter of 5 mm (as indicated by a circle in FIG. 7); and
3) a Blank group, a PLGA/pDA scaffold group and a group of PLGA/pDA scaffold loaded with the PLT-apoVs were set up respectively.

### (3) Micro-Computed Tomography (MicroCT) Analysis

1) 8 weeks later after surgery, all experimental animals were euthanized;
2) the entire skull including implants was surgically removed and fixed with 4% paraformaldehyde;
3) high-resolution Inveon MicroCT (μCT, Siemens) was performed, where experimental parameters included: 80 kV of an X-ray voltage, 500 μA of node current, 1500 ms of exposure time, and 360° of rotation; and
4) multi-modal 3D visualization software was used to reconstruct 3D images and evaluate the osteogenic capability.

FIG. 7 is a MicroCT scan diagram of PLT-apoVs that promote bone defect repair of mesenchymal stem cells. It can be intuitively seen that compared with the Blank group and the PLGA/pDA scaffold group, the group of PLGA/pDA loaded with the human platelet-derived apoptotic vesicles was advantageous in that after implantation into a defect site, more new bone tissues were formed around and in a center of the defect site.

The above are the preferred embodiments of the present application. It should be noted that for those of ordinary skill in the art, they may make several improvements and modifications on the premise without deviating from a principle of the present application, and these improvements and modifications shall fall within the protection scope of the present application.

1.-10. (canceled)

11. A method of promoting osteogenic differentiation of mesenchymal stem cells, comprising culturing the mesenchymal stem cells under the presence of human platelet-derived apoptotic vesicles.

12. The method of claim 11, wherein the mesenchymal stem cells are bone marrow mesenchymal stem cells.

13. The method of claim 11, wherein the mesenchymal stem cells are human mesenchymal stem cells.

14. The method of claim 11, wherein the human platelet-derived apoptotic vesicles promotes osteogenic differentiation of the mesenchymal stem cells by upregulating RUNX2 gene expression of the mesenchymal stem cells.

15. The method of claim 11, wherein the human platelet-derived apoptotic vesicles are biconcave and disc-shaped, with a particle size of 90 nm to 300 nm.

16. The method of claim 11, further comprising preparing the human platelet-derived apoptotic vesicles, comprising:
isolating and purifying human platelets in vitro;
re-suspending the human platelets in a culture solution, and inducing apoptosis of the human platelets under the presence of staurosporine; and
collecting a supernatant and isolating the human platelet-derived apoptotic vesicles from the supernatant by a gradient centrifugation.

17. The method of claim 16, wherein the culture solution is a minimum essential medium α (MEMα) containing 10% fetal bovine serum (FBS) and 1% penicillin-streptomycin dual antibiotics.

18. The method of claim 16, wherein the gradient centrifugation comprises:
centrifuging the supernatant at 4° C. and 800 g for 10 min, to obtain a first centrifugation supernatant;
centrifuging the first centrifugation supernatant at 4° C. and 16,000 g for 30 min, and taking a precipitate to obtain a second centrifugation supernatant to obtain crude apoptotic vesicles (apoVs); and
washing the crude apoVs with sterile phosphate-buffered saline (PBS), and then centrifuging at 4° C. and 16,000 g for 30 min, to obtain the human platelet-derived apoptotic vesicles.

19. A method of repairing a bone defect of a subject, comprising promoting osteogenic differentiation of the mesenchymal stem cells according to the method of claim 11.

20. The method of claim 19, comprising preparing a bone defect repair formulation, comprising:
forming the human platelet-derived apoptotic vesicles on a surface of a polylactic acid-hydroxyacetic acid copolymer (PLGA) scaffold.

21. The method of claim 19, wherein the human platelet-derived apoptotic vesicles are derived from platelets of the subject.

22. The method of claim 20, wherein the PLGA scaffold has a diameter of 4 mm and a height of 2 mm.

23. The method of claim 20, wherein said forming the human platelet-derived apoptotic vesicles on the surface of the PLGA scaffold comprises:
forming polydopamine (pDA) membrane on the PLGA scaffold by soaking the PLGA scaffold in a dopamine solution;
removing unattached dopamine to obtain a PLGA/pDA scaffold; and
soaking the PLGA/pDA scaffold in a solution comprising the human platelet-derived apoptotic vesicles.

24. The method of claim 23, wherein said soaking the PLGA scaffold in the dopamine solution comprises soaking the PLGA scaffold in the dopamine solution and shaking and culturing at 37° C. for 18 hours.

25. The method of claim 23, wherein said removing the unattached dopamine to obtain the PLGA/pDA scaffold comprises:
removing the unattached dopamine by distilled water in an ultrasonic cleaning machine;
sterilizing with 75% ethanol for 1 hour; and
washing with sterile phosphate-buffered saline (PBS) for 3 times.

26. The method of claim 23, wherein a concentration of the dopamine solution is 2 mg/mL.

27. The method of claim 23, wherein a concentration of the human platelet-derived apoptotic vesicles in the solution comprising the human platelet-derived apoptotic vesicles is 500 ng/μL.

28. A bone defect repair formulation, comprising human platelet-derived apoptotic vesicles on a polylactic acid-hydroxyacetic acid copolymer (PLGA) scaffold.

29. The formulation of claim 28, wherein the bone defect repair formulation comprises:
the PLGA scaffold;
a polydopamine (pDA) membrane on the PLGA scaffold;
the human platelet-derived apoptotic vesicles on the pDA membrane.

30. The formulation of claim 28, wherein the human platelet-derived apoptotic vesicles are derived from platelets of a subject.
